(19) United States
(12) Patent Application Publication
(10) Pub. No.: US 2005/0137666 A1
(43) Pub. Date: Jun. 23, 2005

(54) Method and apparatus for a vascular exchange introducer

(76) Inventors: Paul A. Kurth, Santa Barbara, CA (US); Seth J. Worley, Lancaster, PA (US)

Correspondence Address: Daniel L. Dawes;MYERS DAWES ANDRAS & SHERMAN LLP, Suite 1150, 19900 MacArthur Boulevard, Irvine, CA, 92612, US

(21) Appl. No.: 10/977,139

(22) Filed: Oct. 28, 2004

Related U.S. Application Data
(60) Provisional application No. 60/515,936, filed on Oct. 29, 2003.

(51) Int. Cl. A61N 1/05

(52) U.S. Cl. 607/116

(57) ABSTRACT

A method of exchanging vascular introducers comprises the steps of telescopically disposing a guide into a first introducer having a first inner diameter, removing the first introducer from the guide, telescopically disposing a second introducer having a second inner diameter different than the first inner diameter over the guide, and removing the guide. A kit for exchanging cardiac introducers through which a pacemaker lead has been introduced comprises a removable first introducer which is endovascularly implanted into a heart, a guide telescopically disposable into the first introducer, and a second introducer telescopically disposable over the guide to assume the position of the first introducer in the heart.

### RELATED APPLICATIONS

The present application is related to U.S. Provisional Patent Application, Ser. No. 60/515,936, filed on Oct. 29, 2003, which is incorporated herein by reference and to which priority is claimed pursuant to 35 USC 119.

### BACKGROUND OF THE INVENTION

1. Field of the Invention

The invention relates to the field of vascular introducers and in particular to cardiac pacemaker lead introducers and their method of exchange.

2. Description of the Prior Art

Pacemakers are introduced into the heart through an introducer which typically extends from a subclavian insertion site into the targeted heart chamber or in the case of a coronary venous implanted into the coronary sinus. For a variety of different reasons it may become necessary in the course of a pacemaker implant to implant a lead or other instrument through the introducer which has been initially inserted, which lead or other instrument has a larger diameter than the inner diameter of the inserted introducer or a differently shaped introducer might be called for. In general, the hemostatic introducer may be damaged or kinked, the valve may leak, and/or the introducer body may be the wrong shape or diameter as determined by the surgeon at that point in the procedure. In this case the inserted introducer will need to be exchanged for a larger one. However, the insertion of the introducer into the heart typically takes some skill, time and effort and the results of its insertion might be lost, if it were to be simply withdrawn and a larger introducer reinserted into the heart without more. Hence, the benefit of surgical work effort can easily be lost and its reduplication required with the resultant increase in probability of trauma, error, and critical surgical time.

What is needed is some kind of apparatus or method whereby such risk of loss of surgical benefit can be avoided.

### BRIEF SUMMARY OF THE INVENTION

The illustrated embodiment of the invention is defined as a method of exchanging vascular introducers comprising the steps of telescopically disposing a guide into a first introducer having a first inner diameter which has a mating relationship with the first introducer and a second introducer. The first introducer is removed from the guide. The second introducer having a second inner diameter different than the first inner diameter is telescopically disposed over the guide. The guide is then removed.

The method further comprises stabilizing the position of the guide when the second introducer is telescopically disposed thereover.

The step of removing the first introducer comprises removing the first introducer where the first inner diameter is less than the second inner diameter of the second introducer and where telescopically disposing the guide into the first introducer comprises establishing a mating relationship between the guide and the first introducer.

The step of telescopically disposing the second introducer over the guide establishes a mating relationship between the guide and the second introducer, the relative difference in the inner diameter of the first and second introducers allowing for a common mating relationship with the guide.

The method further comprises the step of disposing tubing over the guide prior to the telescopically disposing the second introducer over the guide to provide a mating relationship between the guide and second introducer, when the difference the inner diameter of the second introducer is much larger than the inner diameter of the first introducer, the tubing having a mating relationship with the second introducer and the guide having a mating relationship with the tubing.

The step of removing the first introducer comprises removing the first introducer where the first inner diameter is greater than the second inner diameter of the second introducer and where telescopically disposing the guide into the a first introducer comprises disposing a guide having a mating relationship with the second introducer, but not necessarily with the first introducer.

The step of removing the first introducer from the guide comprises removing the first introducer by telescopically withdrawing the first introducer over the proximal end of the guide. The guide has an envelope which is subsumable within the inner diameter of the first introducer.

The step of removing the first introducer by telescopically withdrawing the first introducer over the proximal end of the guide comprises temporarily configuring the proximal end of the guide to be subsumable within the inner diameter of the first introducer.

The step of temporarily configuring the proximal end of the guide to be subsumable within the inner diameter of the first introducer comprises temporarily deforming a termination on the proximal end of the guide to be telescopically disposable within the inner diameter of the first introducer.

The step of temporarily configuring the proximal end of the guide to be subsumable within the inner diameter of the first introducer comprises temporarily removing a termination on the proximal end of the guide when the first introducer is telescopically removed over the proximal end.

The step of temporarily configuring the proximal end of the guide to be subsumable within the inner diameter of the first introducer comprises attaching a termination on the proximal end of the guide after the first introducer is telescopically has been removed over the proximal end, the guide having no obstructing termination on the proximal end of the guide when the first introducer is telescopically removed over the proximal end of the guide.

The step of telescopically disposing a second introducer having a second inner diameter different than the first inner diameter over the guide comprises telescopically disposing the second introducer by telescopically sliding the first introducer over the proximal end of the guide.

The step of telescopically disposing the second introducer by telescopically sliding the first introducer over the proximal end of the guide comprises temporarily configuring the proximal end of the guide to be subsumable within the inner diameter of the second introducer.

The step of temporarily configuring the proximal end of the guide to be subsumable within the inner diameter of the second introducer comprises temporarily deforming a termination on the proximal end of the guide to be telescopically disposable within the inner diameter of the second introducer.

The step of temporarily configuring the proximal end of the guide to be subsumable within the inner diameter of the second introducer comprises temporarily removing a termination on the proximal end of the guide when the second introducer is telescopically slid over the proximal end.

The step of temporarily configuring the proximal end of the guide to be subsumable within the inner diameter of the second introducer comprises attaching a termination on the proximal end of the guide after the second introducer is telescopically slid over the proximal end. The guide has no obstructing termination on the proximal end of the guide when the second introducer is telescopically slid over the proximal end of the guide.

The invention can also be alternatively characterized as a method of exchanging cardiac introducers through which a pacemaker lead has been introduced into a vascular system of a patient, where a first introducer has been disposed into the vascular system of the patient and a pacemaker lead has been at least in part disposed into the first introducer. The method comprises the steps of removing the pacemaker lead from the first introducer, telescopically disposing a guide into the first introducer to assume the position within the vascular system of the first introducer, removing the first introducer from the guide without loss of the position within the vascular system which had been obtained by the first introducer, telescopically disposing a second introducer over the guide to assume the position of the first introducer in the heart, removing the guide, and telescopically disposing the pacemaker lead or a replacement pacemaker lead through the second introducer to assume a position in the heart initially intended for the pacemaker lead or a replacement pacemaker lead.

The illustrated embodiment of the invention is also defined as an apparatus for exchanging a first introducer having a first inner diameter for a second introducer having a second inner diameter. A guide has an outer diameter such that the guide is telescopically disposable into the first introducer. The second introducer is telescopically disposable over the guide. The relative inner diameters of the first and second introducers with respect to the outer diameter of the guide are such that the guide has a mating relationship with at least the second introducer.

The guide may also have a mating relationship with the first introducer.

The apparatus may further comprise tubing disposable over the guide to facilitate the mating relationship of the guide with the second introducer when the difference in inner diameters of the first and second introducers justifies use of the tubing as a shim. The tubing has a mating relationship with the guide and with the second introducer.

A further embodiment is used in combination with an elongate instrument and where the guide has a longitudinal lumen defined therethrough for telescopic insertion of the elongate instrument. The longitudinal lumen may also be used for injection of fluid therethrough.

In another embodiment the guide has a variable stiffness as a function of longitudinal position along the guide.

In still another embodiment the guide has a predetermined shape to facilitate oriented positioning of the second introducer in the vascular system. The second introducer has a predetermined shape for positioning in the vascular system.

In yet another embodiment the guide further comprises markings disposed thereon to indicate the amount of length of the guide disposed into the vascular system.

The guide may have radioopaque markings to fluoroscopically display its endovascular position.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a partial cross sectional side view of an introducer and a pacemaker lead as inserted into the vascular system of a patient.

FIG. 2 is a partial cross sectional side view of a guide according to the invention which has replaced the introducer of FIG. 1 after the pacemaker has been temporarily removed.

FIG. 3 is a partial cross sectional side view of an exchange introducer disposed over the guide of FIG. 2 according to the invention into the vascular system of the patient.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The invention is drawn to a method and apparatus whereby cardiac introducers may be exchanged in an efficient and beneficial manner. As will be apparent in the following, the invention is characterized by the ability of the exchange system to smoothly mate the guide to the exchange introducers so that the introducers are smoothly disposed into the vascular system without difficulty, with a minimal of additional trauma and without any significant loss in the surgical or anatomical positioning which as been achieved up to that point in the endovascular intervention.

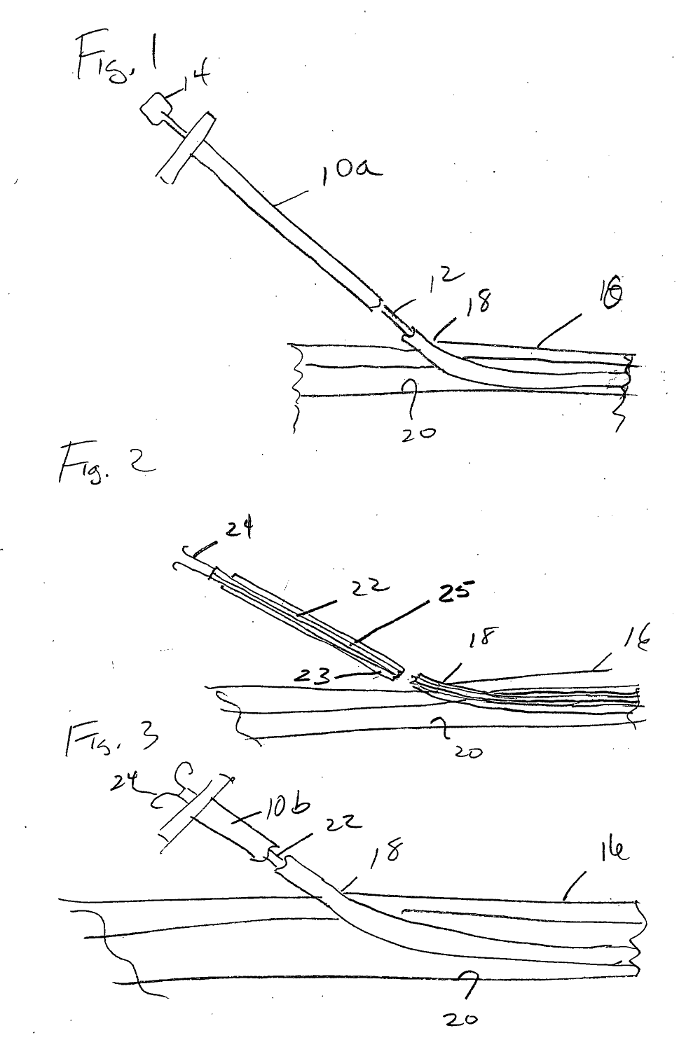

FIG. 1 is a simplified diagram in side partially cross sectional view which shows a partially cut away introducer 10a through which a pacemaker lead 12 has been telescopically disposed. Introducer 10a is inserted into an incision site 18 in the skin 16 into a vein or artery 20, typical the subclavian vein, and has its distal end (not shown) inserted into a desired location in a heart chamber or into the venous system of the heart. Note that lead 12 has a proximal fitting 14 which has a configuration larger than the inner diameter of introducer 10a. Hence introducer 10a is separable by peeling, tearing, cutting, splitting or some other means to allow it to be withdrawn off of lead 12.

Now assume for some reason that lead 12 must be exchanged for a larger diameter lead or instrument of some sort with a larger diameter than can be accommodated by introducer 10a. Introducer 10a cannot be removed without effectively destroying it and lead 12 cannot be removed and the new lead introduced without an appropriately sized and positioned introducer.

A guide 22 as shown in the diagrammatic side cross sectional view is provided according to the invention. Lead 12 is removed from introducer 10a in FIG. 1 leaving introducer 10a in place. Then guide 22 is introduced in the place of lead 12 through introducer 10a. Guide 22 had an outer diameter smaller than the inner diameter of introducer 10a. Then introducer 10a is removed from guide 22 either by separation of introducer 10a or peeling it apart as is normally performed so that it can be easily removed over or around proximal termination 24 of guide 22, which is diagrammatically shown as a flexible pair of finger tabs forming termination 24. Termination 24 of guide 22 may have many different embodiments other than that implied or diagrammatically shown here. Any form which allows an introducer to be proximally disposed over termination 24 can be used and is deemed equivalent for the purposes of the invention. For example, termination 24 may include a bore or small fitting defined in guide 22 to which a handle is later connected after termination 24 has cleared the proximal end of introducer 10b or distal end of introducer 10a. Guide 22 is long enough so that it holds the position in the heart or endovascular system as earlier achieved by the positioning of introducer 10a and/or lead 12.

Then as shown in FIG. 3 a new, larger diameter introducer 10b is slidingly disposed over termination 24 and telescopically disposed over guide 22 to assume the previous position of introducer 10a and/or lead 12. The proximal portion of guide 22 may have sufficient length such that introducer 10b can be completely telescopically disposed over introducer 10b to expose termination 24 before being inserted to any substantial degree or at all into vein 20. Guide 22 can be stabilized during this time by being handheld or gripped with another suitable device at or just above the insertion site 18. Then, termination 24 can then be held by the surgeon or otherwise fixed so that the exchange introducer 10b can be fully inserted into the heart or endovascular system and the position of guide 22 stabilized regardless of any friction between introducer 10b and guide 22 due to any endovascularly imposed bending of introducer 10b and guide 22. Introducer 10b can be slidingly disposed over termination 24 because of the ability of termination 24 to resiliently deform to assume a three dimensional envelope that will pass within both introducers 10a and 10b. Once introducer 10b is in place, guide 22 is removed and the new larger lead or other instrument (not shown) can be implanted through introducer 10b as intended.

It can now be appreciated that according to the invention the guide 22 will be sized to mate with or conform with both the inner diameter and perhaps the shape of introducer 10a and 10b, so that it will smoothly mate with introducer 10b and be conducive to allowing introducer 10b to be easily disposed over guide 22 to take up the former position in the vascular system of introducer 10a, but still not be difficult to insert into introducer 10a just prior to the exchange. In the case of a wire, this would not be possible. For example, if a wire with a substantially smaller, nonconforming or nonmating diameter and shape than both introducers 10a and 10b were disposed in introducer 10a, the exchange of introducer 10b would be subject to the probability of nonoptimal alignment or positioning in the vascular system as it was being inserted over the wire. It is very likely, for example, that the use of a thin wire would not prevent an exchange introducer from hanging up on the coronary valves in the coronary venous system, if that were the portion of the vascular system where the exchange was occurring. Also the closer the outer diameter of guide 22 to the outer diameter of both introducer 10a and 10b, the more likely cutaneous insertion of the exchange introducer 10b will proceed smoothly with minimal trauma to the insertion site.

Thus, according to the invention the relationship of the size and shape of guide 22 to both introducers 10a and 10b is made as close or as similar as possible so that the exchange of introducers 10a and 10b occurs as smoothly and seamlessly as possible. This relationship is defined for the purposes of this specification as a “mating” or “conforming” relationship between guide 22 and introducer 10a and/or introducer 10b or that guide 22 mates or conforms to introducer 10a and/or introducer 10b.

The mating relationship has been described above in the context of an introducer 10a being exchanged for an introducer 10b of the same or a smaller diameter. In this case, the outer diameter of guide 22 is chosen to conform with the smaller introducer 10b and by assumption easily fits within the larger introducer 10a. Introducer 10b then smoothly conforms to introducer 10b and facilitates its vascular placement.

The mating relationship can also be realized when an introducer 10a is exchanged for an introducer 10b of a larger diameter. Guide 22 can clearly be chosen to have an outer diameter that will fit inside both the smaller inner diameter introducer 10a and larger inner diameter introducer 10b. If the diameters of introducer 10a and 10b are too dramatically different, then hollow, flexible tubing 23 having an inner diameter which will accommodate the largest diameter guide 22 that can be disposed in introducer 10a and an outer diameter that is accommodated in or mates with introducer 10b, can be disposed over guide 22 after introducer 10a is removed to provide a conforming or mating fit with the inner diameter of the much larger introducer 10b. Tubing 23 is used in this situation as a shim. It is thus possible that one or more pieces of tubing 23 could be combined and that the inner and outer surfaces of tubing 23 coated or lubricated to allow ease of insertion.

The invention can of course be used in an exchange of the opposite nature. Namely, the guide 22 may have a diameter sufficiently small that introducer 10b may have an inner diameter smaller than introducer 10a and introducer 10b can still be telescopically disposed over guide 22.

While the invention has been described as exchanging introducers of different sizes, it also contemplates exchanging introducers of the same size. For example, a pacemaker lead 12 may become dislodged as the first introducer 10a is being peeled apart or there may have been some other change of circumstance or configuration of the pacemaker lead 12, which requires its reinsertion or replacement. Rather than risk bleeding or air embolism by using a partially peeled introducer 10a without any intact hemostatic valve, the surgeon may decide to withdraw the original pacemaker lead 12, insert the guide 22 through the partially peeled first introducer 10a, replace the first introducer 10a with a second introducer 10b by disposition over the guide 22 in which example the second introducer 10b has the same diameter as the first introducer 10a but with an intact hemostatic valve, remove the guide 22, and then reinsert the original pacemaker lead 12 or insert a new pacemaker lead 12′.

Many alterations and modifications may be made by those having ordinary skill in the art without departing from the spirit and scope of the invention. For example, guide 22 may be fabricated to have a graduated or predetermined profile of stiffness and outer diameter. The proximal portion of guide 22 may be reinforced with or made from relatively stiff plastic or metal fibers or tubing and then thinned and bonded with a more pliable plastic or metallic material to exhibit a gradual change in stiffness or resiliency as a function of longitudinal position according to well understood fabrication techniques. The distal portion of guide 22 can similarly be made even more pliable by being fabricated from a longitudinally increasingly soft or floppy polymeric material or soft metal coil. In this way, guide 22 exhibits sufficient proximal size and rigidity to allow convenient handling and manipulation by the surgeon, but at the distal portion disposed within the heart or coronary vascular system, guide 22 has a substantially smaller diameter and pliability so that it nontraumatically conforms to the tortuous track of the coronary arteries or vessels. In the case where guide 22 is made entirely from a polymer, it is possible to have selected variable durometer defined as a function of longitudinal position along guide 22, or a longitudinal continuous or piecewise continuous gradient of durometer according to the application at hand.

Still further, guide 22 may have a longitudinal lumen 25 defined therethrough to allow for the injection of contrast agents or drugs into the vascular system during the procedure. The longitudinal lumen 25 may also be used for placement of a thin wire either to assist in steering guide 22, for guiding guide 22 or for any kind of vascular intervention which uses a wire for vascular manipulation, electrophysiological stimulation or sensing.

The shape of guide 22 can also be used to actively steer the introducer or not. For example, the first introducer 10a used may have one biased shape or no shape at all, and it is desired in the medical procedure being practiced to insert an introducer which is or can be shaped to access a different position in the vascular system. Guide 22 will then have the new desired shape, which may or may not be so strongly biased in guide 22 that it is impressed upon introducer 10a when guide 22 is inserted into it. In any case, after introducer 10a is removed, guide 22 assumes its biased shape, which may or may not be the same as the final shape of introducer 10b, but which can be used as a means of steering the exchange introducer 10b to its desired position in the vascular system or at least serve as an aid in transitioning the endovascular deployment from the shape of introducer 10a to that of introducer 10b.

Guide 22 can also be provided with markers or graduated rulings on its outside surface so that the amount or length of guide 22 which has been vascularly inserted can be read off guide 22 at a proximal position, either at the insertion site or at proximal location of manipulation.

The invention expressly contemplates that guide 22 will have one or more radioopaque markers to allow or facilitate its fluoroscopic visualization, as typically characterized by a radioopaque tip marker, which marks it most distal position within the vascular system.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

1. A method of exchanging vascular introducers comprising:
telescopically disposing a guide into a first introducer having a first inner diameter which has a mating relationship with the first introducer and a second introducer;
removing the first introducer from the guide;
telescopically disposing the second introducer having a second inner diameter different than the first inner diameter over the guide; and
removing the guide.

2. The method of claim 1 further comprising stabilizing the position of the guide when the second introducer is telescopically disposed thereover.

3. The method of claim 1 where removing the first introducer comprises removing the first introducer where the first inner diameter is less than the second inner diameter of the second introducer and where telescopically disposing the guide into the first introducer comprises establishing a mating relationship between the guide and the first introducer.

4. The method of claim 3 where telescopically disposing the second introducer over the guide establishes a mating relationship between the guide and the second introducer, the relative difference in the inner diameter of the first and second introducers allowing for a common mating relationship with the guide.

5. The method of claim 3 further comprising disposing tubing over the guide prior to the telescopically disposing the second introducer over the guide to provide a mating relationship between the guide and second introducer, when the difference the inner diameter of the second introducer is much larger than the inner diameter of the first introducer, the tubing having a mating relationship with the second introducer and the guide having a mating relationship with the tubing.

6. The method of claim 1 where removing the first introducer comprises removing the first introducer where the first inner diameter is greater than the second inner diameter of the second introducer and where telescopically disposing the guide into the a first introducer comprises disposing a guide having a mating relationship with the second introducer, but not necessarily with the first introducer.

7. The method of claim 1 where the guide has a proximal end with an envelope subsumable within the inner diameter of the first introducer and where removing the first introducer from the guide comprises removing the first introducer by telescopically withdrawing the first introducer over the proximal end of the guide.

8. The method of claim 7 where removing the first introducer by telescopically withdrawing the first introducer over the proximal end of the guide comprises temporarily configuring the proximal end of the guide to be subsumable within the inner diameter of the first introducer.

9. The method of claim 8 where temporarily configuring the proximal end of the guide to be subsumable within the inner diameter of the first introducer comprises temporarily deforming a termination on the proximal end of the guide to be telescopically disposable within the inner diameter of the first introducer.

10. The method of claim 8 where temporarily configuring the proximal end of the guide to be subsumable within the inner diameter of the first introducer comprises temporarily removing a termination on the proximal end of the guide when the first introducer is telescopically removed over the proximal end.

11. The method of claim 8 where temporarily configuring the proximal end of the guide to be subsumable within the inner diameter of the first introducer comprises attaching a termination on the proximal end of the guide after the first introducer is telescopically has been removed over the proximal end, the guide having no obstructing termination on the proximal end of the guide when the first introducer is telescopically removed over the proximal end of the guide.

12. The method of claim 1 where the guide has a proximal end with an envelope subsumable within the inner diameter of the second introducer and where telescopically disposing a second introducer having a second inner diameter different than the first inner diameter over the guide comprises telescopically disposing the second introducer by telescopically sliding the first introducer over the proximal end of the guide.

13. The method of claim 12 where telescopically disposing the second introducer by telescopically sliding the first introducer over the proximal end of the guide comprises temporarily configuring the proximal end of the guide to be subsumable within the inner diameter of the second introducer.

14. The method of claim 13 where temporarily configuring the proximal end of the guide to be subsumable within the inner diameter of the second introducer comprises temporarily deforming a termination on the proximal end of the guide to be telescopically disposable within the inner diameter of the second introducer.

15. The method of claim 13 where temporarily configuring the proximal end of the guide to be subsumable within the inner diameter of the second introducer comprises temporarily removing a termination on the proximal end of the guide when the second introducer is telescopically slid over the proximal end.

16. The method of claim 13 where temporarily configuring the proximal end of the guide to be subsumable within the inner diameter of the second introducer comprises attaching a termination on the proximal end of the guide after the second introducer is telescopically slid over the proximal end, the guide having no obstructing termination on the proximal end of the guide when the second introducer is telescopically slid over the proximal end of the guide.

17. A method of exchanging cardiac introducers through which a pacemaker lead has been introduced into a vascular system of a patient, where a first introducer has been disposed into the vascular system of the patient and a pacemaker lead has been at least in part disposed into the first introducer, comprising the steps of:
removing the pacemaker lead from the first introducer;
telescopically disposing a guide into the first introducer to assume the position within the vascular system of the first introducer;
removing the first introducer from the guide without loss of the position within the vascular system which had been obtained by the first introducer;
telescopically disposing a second introducer over the guide to assume the position of the first introducer in the heart;
removing the guide; and
telescopically disposing the pacemaker lead or a replacement pacemaker lead through the second introducer to assume a position in the heart initially intended for the pacemaker lead or a replacement pacemaker lead.

18. An apparatus for exchanging a first introducer having a first inner diameter for a second introducer having a second inner diameter comprising a guide having an outer diameter such that the guide is telescopically disposable into the first introducer, and such that the second introducer is telescopically disposable over the guide, the relative inner diameters of the first and second introducers with respect to the outer diameter of the guide being such that the guide has a mating relationship with at least the second introducer.

19. The apparatus of claim 18 where the guide also has a mating relationship with the first introducer.

20. The apparatus of claim 18 further comprising tubing disposable over the guide to facilitate the mating relationship of the guide with the second introducer when the difference in inner diameters of the first and second introducers justifies use of the tubing as a shim.

21. The apparatus of claim 20 where the tubing has a mating relationship with the guide and with the second introducer.

22. The apparatus of claim 18 for use in further combination with an elongate instrument and where the guide has a longitudinal lumen defined therethrough for telescopic insertion of the elongate instrument.

23. The apparatus of claim 18 where the guide has a longitudinal lumen defined therethrough for injection of fluid therethrough.

24. The apparatus of claim 18 where the guide has a variable stiffness as a function of longitudinal position along the guide.

25. The apparatus of claim 18 where the guide has a predetermined shape to facilitate oriented positioning of the second introducer in the vascular system.

26. The apparatus of claim 18 where the second introducer has a predetermined shape for positioning in the vascular system.

27. The apparatus of claim 18 where the guide further comprises markings disposed thereon to indicate the amount of length of the guide disposed into the vascular system.

28. The apparatus of claim 18 where the guide further comprises radioopaque markings to fluoroscopically display its endovascular position.
